(19) United States
(12) Patent Application Publication
(10) Pub. No.: US 2002/0065484 A1
(43) Pub. Date: May 30, 2002

(54) Dual lumen subcutaneous injection set for use with a reservoir that has a septum

(76) Inventors: Joel Sterling Douglas, Los Altos Hills, CA (US); Robert Couis Hugo, Gilroy, CA (US)

Correspondence Address: T. GENE DILLAHUNTY, BURNS, DOANE, SWECKER & MATHIS, L.L.P., P.O. BOX 1404, ALEXANDRIA, VA, 22313-1404, US

(21) Appl. No.: 09/933,452

(22) Filed: Aug. 20, 2001

Related U.S. Application Data
(60) Provisional application No. 60/250,264, filed on Nov. 29, 2000.

(51) Int. Cl. A61M 31/00

(52) U.S. Cl. 604/93.01

(57) ABSTRACT

The present invention relates to a disposable injection set that offers the ability for the patient to infuse from two prefilled medication cartridges without compromising the sterility of medication to be infused. Also, utilization of prefilled medication cartridges to provide a source of infusion material makes it easy and convenient to use by the average patient who must inject themselves with a varying mixture of insulin or medication.

The present application is related and claimed priority to U.S. application Ser. No. 60\250,264, filed Nov. 29, 2000, by Joel Douglas and Robert Hugo, Jr., the entire content is incorporated by reference herein.

### BACKGROUND OF THE INVENTION

1. Field of the Invention

The present invention relates to medical connectors and infusion sets.

2. Brief Description of the Related Art

In general, whenever a therapeutic fluid is to be delivered subcutaneously to a patient from an external source, a passageway, such as that provided by a hollow needle or other type of cannula or catheter device, must be first inserted through the skin of the patient in order to provide a passageway or channel through which the fluid may pass from its source external to the patient to the desired subcutaneous location under the skin of the patient. Once this passageway has been installed, the desired infusion system may be used in conjunction with an appropriate catheter connecting the external source of fluid with the passageway leading to the subcutaneous delivery point to deliver the fluid to the patient at an appropriate delivery rate.

Unfortunately, several problems associated with infusing fluids into the patient as described above are usually encountered. Most patients must infuse two medications at the same time. This is a typical therapy for most people with diabetes who infuse both long acting and fast acting insulin mixes. Additionally, most systems require the patient to use a syringe that is fitted with a thread or luer fitting. The patient must fill the syringe and attach the catheter to it. However, in doing so the medication can become contaminated and is therefore no longer sterile. Non-sterile solutions can lead to infection. In addition the inconvenience of having to fill the syringe in the first place, and then attach and prime the catheter is difficult for many patients. In addition, the connecting tubes for subcutaneous infusion sets customarily are made from low density polyethylene (LDPE), polypropylene or co-extruded PVC and LDPE. These materials prevent the degradation of the medication due to CO² contamination.

In the early 1990's prefilled insulin cartridges were first introduced by insulin manufacturing companies for use in insulin pens. These cartridges are prefilled and their use is very convenient for the patient. The difficulty is that the prefilled cartridges need to have a septum to allow the medication to remain sterile and they contain only one type of insulin. Even with the new premixed insulin's, such as the Novo Nordisk 70/30 mix, that are sold in a prefilled cartridge are not sufficient because only a minority of patients take this particular proportion of insulin mix. Most patients take a variable mixture depending on the state of glycimic control that the patient is in. This variable mix can range fro 90/10 to 60/40.

The prior art has attempted to satisfy the problems associated with multiple subcutaneous delivered drugs. Typical solutions include and the entire content of each disclosure is incorporated by reference herein.

U.S. Pat. No. 4,318,402, Vaillancourt, describes a liquid infusion catheter assembly comprised of a hub and a first hollow catheter connected at its proximal end, and being in fluid communication with, the hub. This catheter has at least one fluid outlet opening. A second hollow catheter, extending from, but being out of fluid communication with, the hub surrounds the first catheter with a space there between. This second catheter extends distally farther than the first catheter and has at least one hole through its surface where it surrounds the first catheter, and has a fluid outlet opening in the portion extending beyond the first catheter.

U.S. Pat. No. 4,257,416, Prager, describes a multi-channel venipuncture infusion set for simultaneously dispensing multiple, intravenous solutions, parenteral fluids and drugs, and which also permits withdrawal of blood samples without disconnecting the infusion set from the dispensing sources and without removing or replacing the venipuncture needle in the patient.

U.S. Pat. No. 4,313,440, Ashley, describes a multipurpose double barrel syringe and method of manufacture.

U.S. Pat. No. 4,886,495, Reynolds, describes a vial-based prefilled syringe system for one or two component medicaments

U.S. Pat. No. 5,032,117, Motta describes a Tandem syringe for the gravity-feed intravenous administration of drugs.

U.S. Pat. No. 5,797,869 Martin, et al. describes a multiple lumen catheter.

One solution that has been proposed to the problem has been through the use of two different insulin pens to inject differing amounts of insulin as need. However, this requires the patients to inject themselves twice and many patients do not view injections as a pleasant experience. A second method is to fill a single syringe with the two different medications or insulins into a single syringe. The elimination of the multiple injections for multiple insulin or medication types is desirable. Additionally, eliminating the problem of having to mix the insulin into a single syringe is also desirable.

It is thereby apparent that there exists a substantial need for an injection set for delivery of two types of insulin or other fluids through a single passage into the body.

### SUMMARY OF THE INVENTION

According to a first exemplary embodiment, a medication infusion set comprised of a dual lumen tube having an exterior surface, a proximal end, a distal end, and two interior parallel lumens, extending between the proximal and distal ends. The connecting tubes for subcutaneous infusion sets is made from either low density polyethylene (LDPE) , polypropylene or co-extruded PVC and LDPE. These materials prevent the degradation of the medication due to CO² contamination. Two rigid cannulas each having a proximal end, a distal end, and a lumen extending between the proximal and distal ends, the cannulas proximal ends are positioned in the proximal end of each parallel lumen, and the proximal end of each lumen is fixtured in an individual housing so the rigid cannula positioned in the lumen protrudes from the proximal end of the housing. Each housing is adapted for attachment to a prefilled medication cartridge. The distal ends of each of the parallel lumens are positioned in a second housing that is fixtured so as to merge the two lumens into single indwelling catheter positioned at the distal end of the second housing and assembly. The proximal ends of each of the dual lumens provides a direct communication between the proximal end which pierces the septum of each attached prefilled cartridge and the distal end containing a single indwelling cannula.

Still other objects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to preferred embodiments of the apparatus and method, given only by way of example, and with reference to the accompanying drawings, in which:

FIG. 1 illustrates a plan view of a first embodiment of an infusion set and connectors in accordance with the present invention;

FIG. 2 illustrates a longitudinal cross-sectional view of the set distal end;

FIG. 3 a plan view, of the proximal fixtures;

### DESCRIPTION OF THE PREFERRED EMBODIMENTS

Referring to the drawing figures, like reference numerals designate identical or corresponding elements throughout the several figures.

The present invention relates to a disposable injection set that offers the ability for the patient to infuse from two prefilled insulin cartridges without compromising the sterility of medication to be infused. Also, utilization of prefilled medication cartridges as a source of infusion material makes it easy and convenient for use by the average patients who must inject themselves with varying mixtures of insulin. The utilization of a cannula to pierce the septum of the medication cartridge provides the sterile means for connecting the catheter to the individual cartridges and by combining it with the soft or flexible catheter with needles fixed on both ends permits the development of a convenient and sterile infusion device capable of delivering two different medications at the same time. The cannula located at the distal end that is inserted into the skin of a patient is held at its insertion location with a low profile holding pad or housing. The pad or housing has merging tubes that merge the two communication paths of the two connecting parallel lumens. The connecting tubes for subcutaneous infusion sets customarily are made from either low density polyethylene (LDPE), polypropylene or co-extruded PVC and LDPE. These materials prevent the degradation of the medication due to CO² contamination. The merging channel must be designed to provide a minimum dead volume so that the selected amount of medication delivered is approximately the delivered amount and therefore, the remaining medication in the indwelling cannula and associated channels in the holding pad has been designed to be small. It is assumed, based on current therapy, that a dead space that contributes less than one unit error is sufficient. To insure that the merging flow is not turbulent and that the dead volume for each merged channel is minimized the design must meet certain criteria.

The opposite end has two threaded connectors with a cannula fixed in it. Each connector permits communication between the fluid in the reservoirs and the indwelling catheter. The cannula on the threaded connectors is positioned so that the tubing individual lumens of the catheter is fixed over the cannula in each threaded connector and attached by adhesive to the separate catheter tubing lumen. This is done for each of the two lumens. The adhesive is selected from a set of epoxies or solvent adhesive that are capable of bonding to the soft tubing. The adhesive needs to create the appropriate bond and be capable of being sterilized. The solvent adhesive is applied to the outer surface of the tube and inserted into the connector or holding pad to create an adequate sealed assembly.

The dimensions of the associated merging channels are designed so as to minimize turbulent flow and dead volume. To insure these requirements they need to be designed per the following criteria which is base on the piercing cannula dimensions. For a device with cannula to pierce the septum of the medication cartridge with an inner diameter less than 0.016 inches and a length no to exceed 1.5 inches the mixing channel size to minimize turbulence and dead volume has a minimum id of 0.016 inches and a maximum length of 0.5 inches. For a device with cannula to pierce the septum of the medication cartridge with an inner diameter less than 0.030 inches and a length not to exceed 2 inches the mixing channel size to minimize turbulence and dead volume has a minimum id of 0.020 inches and a maximum length of 0.5 inches. These dimensions will insure that the piercing cannula has the largest resistance to fluid flow and that the mixing channels present the smallest volume possible without increasing the resistance to fluid flow.

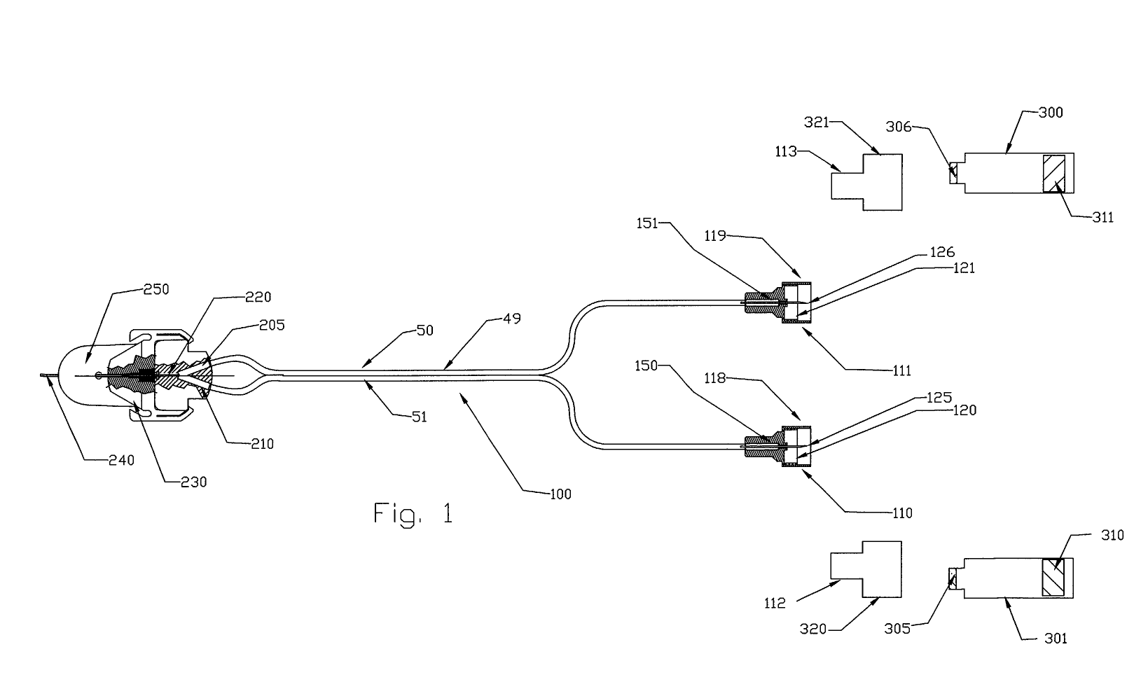

Turning now to the drawing, FIG. 1 illustrates a plan view of the first embodiment of a catheter infusion set 100 of an infusion apparatus in accordance with the present invention. Generally, the apparatus 100 includes a medication delivery device, into which medication ampoules 300 and 301 are releasably installed, and a catheter infusion set 100. The medication delivery device can be similar in construction to any of numerous such devices which receive a medication ampoule 300 and 301, including “pen” type injectors, programmable medication pumps, and those described in U.S. application Ser. No. 60/156,535, filed Sep. 29, 1999, U.S. application Ser. No. 60/170,570, filed Dec. 13, 1999, U.S. application Ser. No. 60/177,762, filed Jan. 24, 2000, and Attorney Docket No. 032994-012, filed Sep. 29, 2000, “Reusable Medication Delivery Device”, to Joel Douglas et al., the entire contents of each of which are incorporated by reference herein. The difference is that the medication delivery device holds two medication ampoules 300 and 301 instead of the customary single medication ampoule.

The medication delivery device includes holders 320 and 321 which releasably holds the medication ampoule 300 and 301 therein so that the ampoule's septum 305 and 306 are exposed and accessible to be pierced and accessed in a known fashion. Similarly, the pistons 310 and 311 of the ampoule 300 and 301 are accessible so that it can be moved to expel medication from the ampoules, again in a known fashion. The holders 320 and 321 include external threads 112 and 113 so that a medical connector 110 and 111, described in greater detail below, can be releasably attached to the device to access the ampoule's contents through the septum's 305 and 306.

The catheter infusion set 100 includes proximal connectors 110 and 111 having a cylindrical collar or shrouds 118 and 119, which has internal threads 120 and 121 which mate with external threads 112 and 113. A piercing element 125 and 126, e.g., needles, extends proximally from the connectors 110 and 111. A distal catheter, cannula, needle, or other tubular element/ catheter 240 extends from the distal end of the set 100, and is in fluid communication with the piercing elements 125 and 126 via connecting tube 49 comprised of lumens 50 and 51, channels 205 and 210 and infusion needle 220. Connecting tube 49 forms the walls of lumens 50 and 51 and can be made from low density polyethylene (LDPE), polypropylene or co-extruded PVC and LDPE. These materials prevent the degradation of the medication due to CO² contamination. As can be seen in FIG. 1, the catheter 240 extends from pad 230 so that the catheter can reside comfortably in the subcutaneous tissue layers of a patient. A holding pad 250 is connected to the pad 230 by any of numerous ways, preferably by an adhesive. At the proximal end of the set 100, the tube 49 is secured to a distal portion 150 and 151 of the connector 110 and 111.

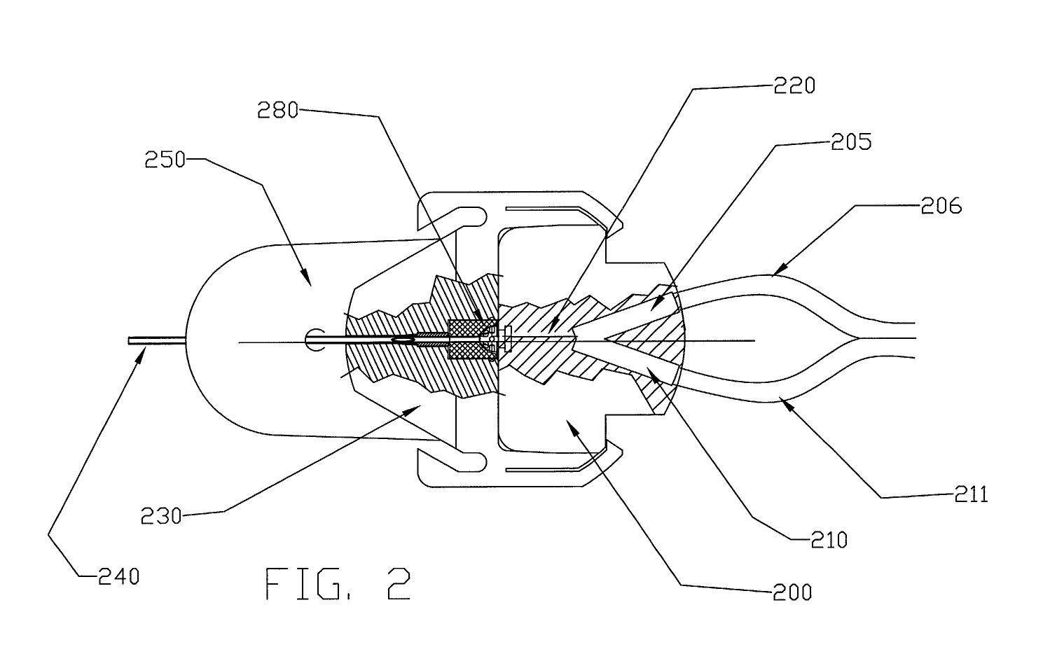

Turning now to the drawing, FIG. 2 illustrates a plan view the first embodiment catheter infusion set 100 distal end of an infusion apparatus in accordance with the present invention. Connecting hub 200 has channels 205 and 210 and the distal ends of lumens 50 and 51, which are part of connecting tube 49, are bonded to the channels. Connecting tube 49 distal ends are shown as 206 and 211, 206 is bonded to channel 205 and 211 is bonded to 210. The diameters of channels 205 and 210 are sized to minimize their volume that reduces the medication that is contained in the channels after infusion. The channels 205 and 210 are in communication with infusion needle 220 and it pierces septum 280 that seals the cannula 240 in infusion hub 230.

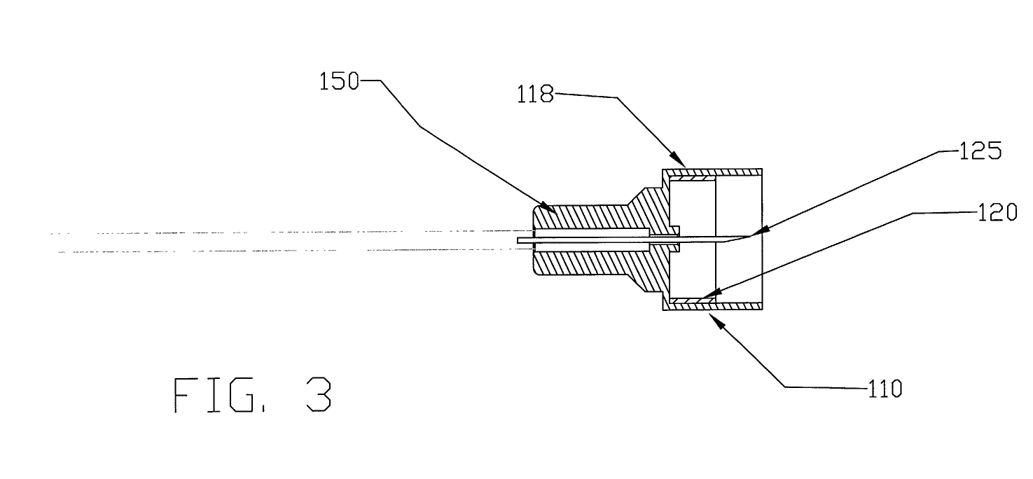

Turning now to the drawing figure, FIG. 3 illustrates a plan view of the first embodiment catheter infusion set 100 proximal ends of an infusion apparatus in accordance with the present invention. Proximal end connectors 110 and 111 are identical. Looking at connector 110 specifically. The holder 320 includes external thread 112 so that a medical connector 110 can be releasably attached to the device to access the ampoule's contents through the septum 305.

The catheter infusion set 100 includes proximal connector 110 having a cylindrical collar or shrouds 118, which have internal threads 120 that mate with external threads 112. A piercing element 125, e.g., needle, extends proximally from the connector 110 and is used to pierce septum 305 when the connector 110 is attached to holder 320 which positions piercing element 125 so that it can pierce septum 305.

The operation of the exemplary embodiment of the present invention described above and illustrated in FIGS. 1,2 and 3 will now be described with reference thereto. A user or patient inserts a medication ampoule 300 and 301, such an insulin cartridge, into the delivery device, with the ampoule's septum's 305 and 306 accessible through the device. The collars 118 and 119 are screwed or otherwise placed over the end of the device so that the piercing elements 125 and 126 pierces the septum's 305 and 306 and places the interior of the ampoule in fluid communication with the cannula 49 connecting tubes 50 and 51. The user can then prime the set 100, if desired. If the set 100 is already in place subcutaneously, the user manipulates the delivery device to move the pistons 310 and 311 separately to administer the correct dosage of both medications and to deliver those amounts of medication to the patient through the cannula 124.

While the invention has been described in detail with reference to preferred embodiments thereof, it will be apparent to one skilled in the art that various changes can be made, and equivalents employed, without departing from the scope of the invention.

What is claimed is:

1. A medication infusion set comprising:
a base tube having an exterior surface, a proximal end, a distal end, and a first and second inner lumen extending between the proximal and distal ends, the base tube formed of a material selected from the group consisting of polyethylene and polypropylene;
a first rigid cannula having a proximal end, a distal end, and a lumen extending between the proximal and distal ends, the cannula proximal end positioned in of the first base tube lumen at the proximal end forming a fluid communication path from rigid cannula proximal end to the first inner lumen of the base tube;
a second rigid cannula having a proximal end, a distal end, and a lumen extending between the proximal and distal ends, the cannula proximal end positioned in of the second base tube lumen at the proximal end forming a fluid communication path from rigid cannula proximal end to the second inner lumen of the base tube;
an infusion catheter and infusion hub having merging chamber at the distal end of the base tubes that directs the fluid from the first and second inner lumen to the infusion cannula in the distal end of the infusion hub.

2. A medication infusion set in accordance with claim 1, further comprising:
at least separate two connectors each including a cylindrical collar having a hollow interior, a closed end, and an open end, the distal end of the rigid cannula extending through the collar closed end and into each of the hollow interior lumen of the base tube; and
a solvent based adhesive bonding the connector to the base tube individual cannula lumens at the proximal end of the base tube.

3. A medication infusion set in accordance with claim 1, having an infusion catheter and infusion hub further comprising:
a cannula having a proximal end, a distal end, and a lumen extending between the proximal end and distal ends, the cannula proximal end in fluid communication with a merging channel in the proximal end of the hub that is in communication with the base tube distal end lumens such that one lumen is bonded to first channel of the merging channel and the second lumen is bonded to the second channel of the merging channel.

4. An infusion hub designed in accordance with claim 1, with a fluidic merging means that is designed such that the merging channels size are defined as follows for a device with cannula to pierce the septum of the medication cartridge at the proximal end of the base tube lumen with an inner diameter less than 0.016 inches and a length no to exceed 1.5 inches the mixing channel size has a minimum id of 0.016 inches and a maximum length of 0.5 inches. An infusion hub designed in accordance with claim 1, with a fluidic merging means that is designed such that the merging channels size are defined as follows for a device with cannula to pierce the septum of the medication cartridge at the proximal end of the base tube lumen with an inner diameter less than 0.030 inches but greater than 0.016 inches and a length no to exceed 2 inches the mixing channel size has a minimum id of 0.020 inches and a maximum length of 0.5 inches.
